(19) United States
(12) Patent Application Publication
(10) Pub. No.: US 2011/0046106 A1
(43) Pub. Date: Feb. 24, 2011

(54) ARYL SULFONAMIDES AS EFFECTIVE ANALGESICS

(75) Inventors: Norbert Hauel, Schemmerhofen (DE); Angelo Ceci, Mittelbiberach (DE); Henri Doods, Warthausen (DE); Iris Kauffmann-Hefner, Attenweiler (DE); Ingo Konetzki, Aachen-Oberforstbach (DE); Annette Schuler-Metz, Ulm (DE); Rainer Walter, Biberach (DE)

Correspondence Address: MICHAEL P. MORRIS;BOEHRINGER INGELHEIM USA CORPORATION, 900 RIDGEBURY ROAD, P. O. BOX 368, RIDGEFIELD, CT, 06877-0368, US

(73) Assignee: BOEHRINGER INGELHEIM INTERNATIONAL GMBH, Ingelheim am Rhein (DE)

(21) Appl. No.: 12/865,436

(22) PCT Filed: Feb. 5, 2009
(86) PCT No.: PCT/EP09/00768

§ 371 Date: Oct. 12, 2010

(30) Foreign Application Priority Data
Feb. 6, 2008 (EP) 08101319.5

(51) Int. Cl. A61K 31/397; C07D 403/06; C07D 401/06; A61K 31/454; C07D 401/14; C07D 211/06; A61K 31/4453; C07D 241/04; A61K 31/495; A61K 31/4545; A61P 29/00

(52) U.S. Cl. 514/210.2; 548/518; 546/208; 514/326; 546/233; 514/315; 544/400; 514/252.12; 546/189; 514/316

(57) ABSTRACT

The present invention relates to compounds of general formula I

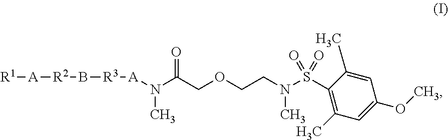

wherein A, B, R¹, R² and R³ are defined as in claim 1, the enantiomers, the diastereomers, the mixtures and the salts thereof, particularly the physiologically acceptable salts thereof with organic or inorganic acids or bases, which have valuable properties, the preparation thereof, the pharmaceutical compositions containing the pharmacologically effective compounds, the preparation thereof and the use thereof.

The present invention relates to compounds of general formula I

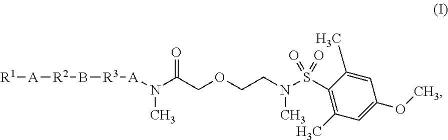

wherein A, B, R¹, R² and R³ are defined as hereinafter, the enantiomers, the diastereomers, the mixtures and the salts thereof, particularly the physiologically acceptable salts thereof with organic or inorganic acids or bases, which have valuable properties, the preparation thereof, the pharmaceutical compositions containing the pharmacologically effective compounds, the preparation thereof and the use thereof.

### DETAILED DESCRIPTION OF THE INVENTION

In the above general formula I in a first embodiment

A denotes
(a) a bond or
(b) a C₁₋₃-alkylene group,
   B denotes
(a) a bond,
(b) a C₁₋₃-alkylene or
(c) a —C(O)— group,
   R¹ denotes
(a) H,
(b) C₁₋₃-alkyl,
(c) C₃₋₆-cycloalkyl,
(d) a 5- or 6-membered, saturated aza-heterocycle or
(e) a 5- or 6-membered, saturated oxa-heterocycle,
   R² denotes
(a) C₁₋₃-alkylene,
(b) a 4- to 6-membered, saturated aza-heterocycle, or
(c) a 6- to 7-membered, saturated diaza-heterocycle,
   R³ denotes
(a) C₂₋₄-alkenylene,
(b) C₂₋₄-alkynylene or
(c) a 5- to 6-membered, saturated aza-heterocycle, wherein additionally a methylene group may be substituted by a carbonyl group,
   the enantiomers, the diastereomers, the mixtures and the salts thereof, particularly the physiologically acceptable salts thereof with organic or inorganic acids or bases.

A second embodiment of the present invention comprises the compounds of the above general formula I, wherein

A denotes a bond or a —CH₂— group,
B denotes a bond, a —CH₂— or —C(O)— group,
R¹ denotes H, H₃C— or a group selected from

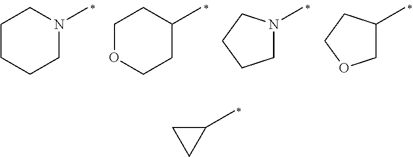

R² denotes a group selected from

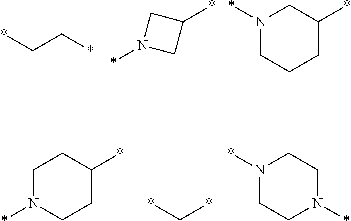

and
R³ denotes a group selected from

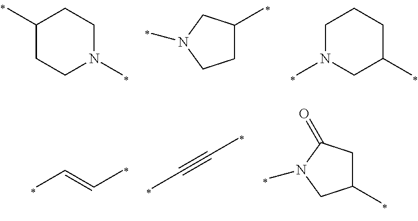

the enantiomers, the diastereomers, the mixtures and the salts thereof, particularly the physiologically acceptable salts thereof with organic or inorganic acids or bases.

The following are mentioned as examples of most particularly preferred compounds of the above general formula I:

| Example | Structure |
|---|---|
| (1) | 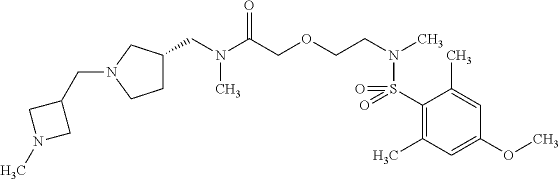 |
| (2) | 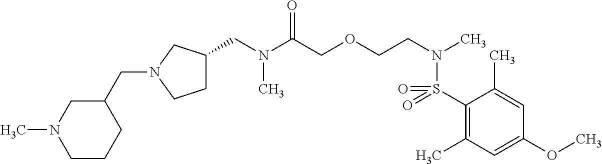 |
| (3) | 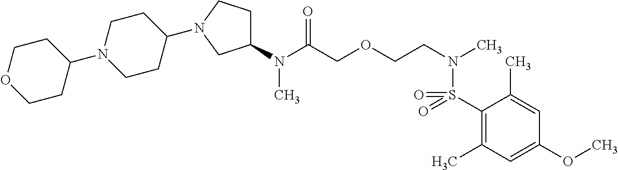 |
| (4) | 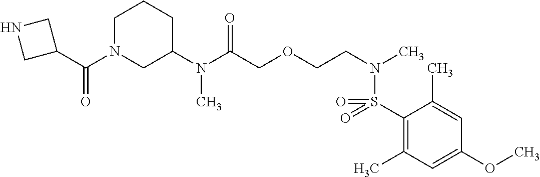 |
| (5) | 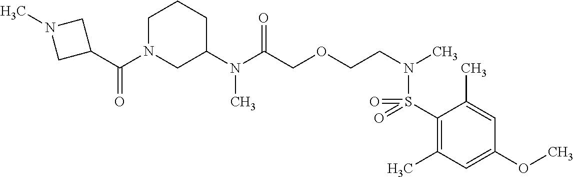 |
| (6) | 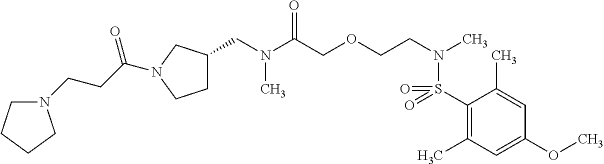 |
| (7) | 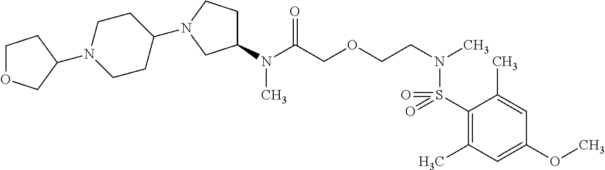 |
| (8) | 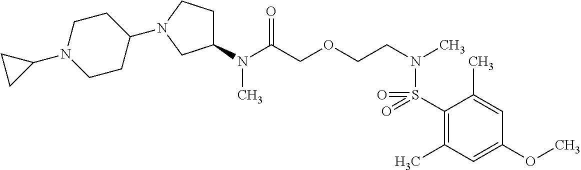 |
| (9) | 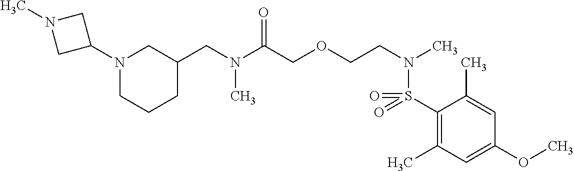 |
| (10) | 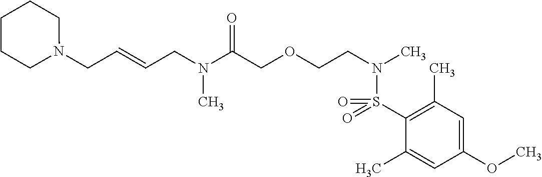 |
| (11) | 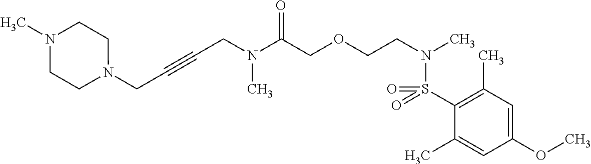 |
| (12) | 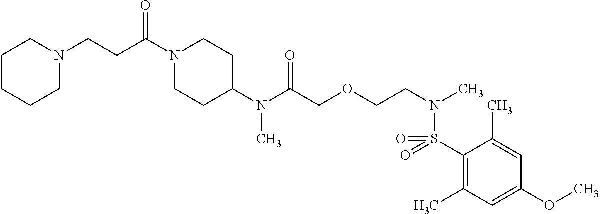 |
| (13) | 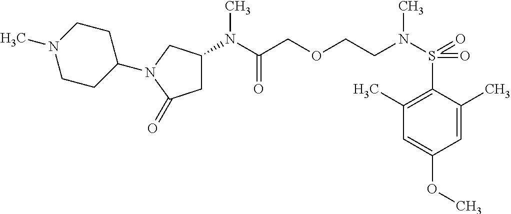 |

the enantiomers, the diastereomers, the mixtures and the salts thereof, particularly the physiologically acceptable salts thereof with organic or inorganic acids or bases.

### TERMS AND DEFINITIONS USED

Within the scope of this application, in the definition of possible substituents, these may also be represented in the form of a structural formula. If present, an asterisk (*) in the structural formula of the substituent is to be understood as being the linking point to the rest of the molecule.

The subject-matter of this invention also includes the compounds according to the invention, including the salts thereof, wherein one or more hydrogen atoms, for example one, two, three, four or five hydrogen atoms, are replaced by deuterium.

By the term “C₁₋₃-alkyl” (including those which are part of other groups) are meant alkyl groups with 1 to 3 carbon atoms. Examples include: methyl, ethyl, n-propyl and iso-propyl. The following abbreviations may also optionally be used for the above-mentioned groups: Me, Et, n-Pr or i-Pr. Unless stated otherwise, the definition propyl includes all the possible isomeric forms of the group, such as, for example, propyl, n-propyl and iso-propyl.

Moreover, the terms mentioned above also include those groups wherein each methylene group may be substituted by up to two fluorine atoms and each methyl group may be substituted by up to three fluorine atoms.

By the term “C₁₋₃-alkylene” are meant branched and unbranched alkylene groups with 1 or 3 carbon atoms. Examples include: methylene, ethylene, ethan-1,1-diyl and propylene. Unless stated otherwise, the definition propylene encompasses all the possible isomeric forms with the same number of carbons, for example 1-methylethylene.

Moreover, the terms mentioned above also include those groups wherein each methylene group may be substituted by up to two fluorine atoms.

By the term “C₂₋₄-alkenylene” (including those which are part of other groups) are meant branched and unbranched alkenylene groups with 2 to 4 carbon atoms. Examples include: ethenylene, propenylene, 1-methylethenylene, butenylene, 1-methylpropenylene, 1,1-dimethylethenylene or 1,2-dimethylethenylene. Unless stated otherwise, the definitions propenylene and butenylene include all the possible isomeric forms of the groups in question with the same number of carbons. Thus, for example, propenyl also includes 1-methylethenylene and butenylene includes 1-methylpropenylene, 1,1-dimethylethenylene, 1,2-dimethylethenylene.

By the term “C₂₋₄-alkynylene” (including those which are part of other groups) are meant branched and unbranched alkynylene groups with 2 to 4 carbon atoms. Examples include: ethynylene, propynylene, 1-methylethynylene, butynylene, 1-methylpropynylene, 1,1-dimethylethynylene or 1,2-dimethylethynylene. Unless stated otherwise, the definitions propynylene and butynylene include all the possible isomeric forms of the groups in question with the same number of carbons. Thus, for example, propynyl also includes 1-methylethynylene and butynylene includes 1-methylpropynylene, 1,1-dimethylethynylene, 1,2-dimethylethynylene.

By the term “C₃₋₆-cycloalkyl” (including those which are part of other groups) are meant cyclic alkyl groups with 3 to 6 carbon atoms. Examples include: cyclopropyl, cyclobutyl, cyclopentyl or cyclohexyl. Unless otherwise stated, the cyclic alkyl groups may be substituted by one or more groups selected from among methyl, ethyl, iso-propyl, tert-butyl, hydroxy, fluorine, chlorine, bromine and iodine.

By the term “saturated aza-heterocycles” are meant four-, five- or six-membered heterocyclic rings which contain a nitrogen atom. The ring is attached to the rest of the molecule either through the nitrogen atom or through the nitrogen atom and a carbon atom. Examples include:

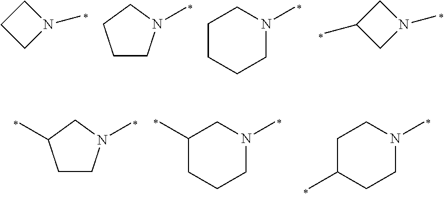

By the term “saturated diaza-heterocycles” are meant six- or seven-membered heterocyclic rings which contain two nitrogen atoms. The ring is attached to the rest of the molecule through the two nitrogen atoms. Examples include:

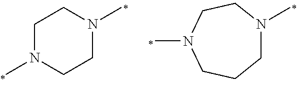

By the term “saturated oxa-heterocycles” are meant five- or six-membered heterocyclic rings which contain an oxygen atom. The ring is attached to the rest of the molecule through a carbon atom. Examples include:

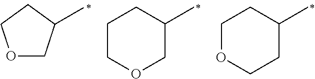

If they contain suitable basic functions, for example amino groups, compounds of general formula I may be converted, particularly for pharmaceutical use, into the physiologically acceptable salts thereof with inorganic or organic acids. Examples of inorganic acids for this purpose include hydrobromic acid, phosphoric acid, nitric acid, hydrochloric acid, sulphuric acid, methanesulphonic acid, ethanesulphonic acid, benzenesulphonic acid or p-toluenesulphonic acid, while organic acids that may be used include malic acid, succinic acid, acetic acid, fumaric acid, maleic acid, mandelic acid, lactic acid, tartaric acid or citric acid. In addition, any tertiary amino groups present in the molecule may be quaternised. Alkyl halides are used for the reaction. According to the invention methyl iodide is preferably used for the quaternisation.

In addition, the compounds of general formula I, if they contain suitable carboxylic acid functions, may if desired be converted into the addition salts thereof with inorganic or organic bases. Examples of inorganic bases include alkali or alkaline earth metal hydroxides, e.g. sodium hydroxide or potassium hydroxide, or carbonates, ammonia, zinc or ammonium hydroxides; examples of organic amines include diethylamine, triethylamine, ethanolamine, diethanolamine, triethanolamine, cyclohexylamine or dicyclohexylamine.

The compounds according to the invention may be present as racemates, provided that they have only one chiral element, but may also be obtained as pure enantiomers, i.e. in the (R) or (S) form.

However, the application also includes the individual diastereomeric pairs of antipodes or mixtures thereof, which are obtained if there is more than one chiral element in the compounds of general formula I, as well as the individual optically active enantiomers of which the above-mentioned racemates are made up.

The invention relates to the compounds in question, optionally in the form of the individual optical isomers, mixtures of the individual enantiomers or racemates, in the form of the tautomers as well as in the form of the free bases or the corresponding acid addition salts with pharmacologically acceptable acids—such as for example acid addition salts with hydrohalic acids—for example hydrochloric or hydrobromic acid—or organic acids—such as for example oxalic, fumaric, diglycolic or methanesulphonic acid.

### Methods of Preparation

According to the invention the compounds of general formula I are obtained by methods known per se, for example by the following methods:

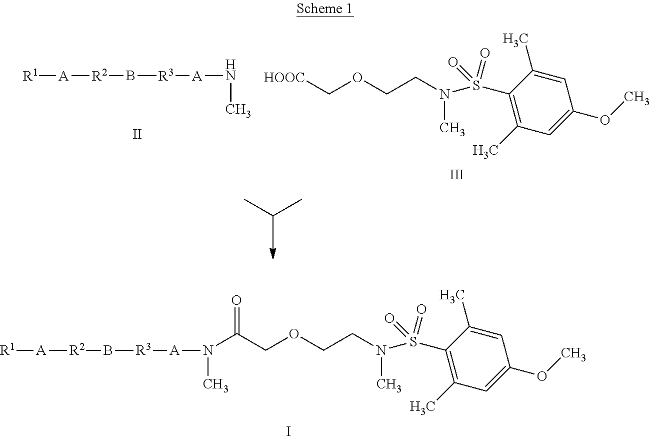

The amine components of general formula II, wherein all the groups are as hereinbefore defined, are prepared using methods known from the literature.

The linking of a carboxylic acid of general formula III shown in Scheme 1, wherein all the groups are as hereinbefore defined, with an amine of general formula II, wherein all the groups are as hereinbefore defined, forming a carboxylic acid amide of general formula I, wherein all the groups are as hereinbefore defined, may be carried out using conventional methods of amide formation.

The coupling is preferably carried out using methods known from peptide chemistry (cf. e.g. Houben-Weyl, Methoden der Organischen Chemie, Vol. 15/2), for example using carbodiimides such as e.g. dicyclohexylcarbodiimide (DCC), diisopropyl carbodiimide (DIC) or ethyl-(3-dimethylaminopropyl)-carbodiimide, O-(1H-benzotriazol-1-yl)-N,N-N′,N′-tetramethyluronium hexafluorophosphate (HBTU) or tetrafluoroborate (TBTU) or 1H-benzotriazol-1-yl-oxy-tris-(dimethylamino)-phosphonium hexafluorophosphate (BOP). By adding 1-hydroxybenzotriazole (HOBt) or 3-hydroxy-4-oxo-3,4-dihydro-1,2,3-benzotriazine (HOObt) the reaction speed can be increased. The couplings are normally carried out with equimolar amounts of the coupling components as well as the coupling reagent in solvents such as dichloromethane, tetrahydrofuran, acetonitrile, dimethyl formamide (DMF), dimethyl acetamide (DMA), N-methylpyrrolidone (NMP) or mixtures thereof and at temperatures between −30° C. and +30° C., preferably −20° C. and +25° C. If necessary, diisopropylethylamine (DIPEA) (Hünig base) is preferably used as an additional auxiliary base.

### Description of the Method of Bradykinin BK1-Receptor Binding

CHO cells stably expressing the rat BK1 receptor are cultivated in Dulbecco's modified medium. The medium from confluent cultures is removed and the cells are washed with PBS buffer, scraped off and isolated by centrifugation. The cells are then homogenized in suspension and the homogenate is centrifuged and resuspended. The protein content is determined and the membrane preparation obtained in this manner is then frozen at −80° C.

After thawing, 200 μl of the homogenate (50 to 100 μg of proteins/assay) are incubated at room temperature with 1 to 5 nM of kallidin (DesArg10, Leu9), [3,4-prolyl-3,43H(N)] and increasing concentrations of the test substance in a total volume of 250 μl for 120 minutes. The incubation is terminated by rapid filtration through GF/B glass fibre filters which had been pretreated with polyethyleneimine (0.3%). The protein-bound radio-activity is measured in a TopCount NXT (Perkin Elmer/Packard). Non-specific binding is defined as radioactivity bound in the presence of 1.0 μM Lys-Des-Arg9-bradykinin. The concentration/binding curve is analysed using a computer-assisted nonlinear curve fitting. The Kᵢ which corresponds to the test substance is determined using the data obtained in this manner.

To demonstrate that the compounds of general formula I with different structural elements show good bradykinin-B1-receptor antagonistic effects, the following Table gives the Kᵢ values obtained according to the test method described above. It is pointed out that the compounds were selected for their different structural elements and not in order to emphasise specific compounds:

| Example | Kᵢ [nM] |
|---|---|
| (3) | 24.7 |
| (6) | 181 |
| (8) | 23.5 |

### Indications

By virtue of their pharmacological properties, the novel compounds and their physiologically acceptable salts are suitable for treating diseases and symptoms of diseases caused at least to some extent by stimulation of bradykinin-B1 receptors.

In view of their pharmacological effect the substances are suitable for the treatment of

(a) acute pain such as e.g. toothache, peri- and postoperative pain, traumatic pain, muscle pain, the pain caused by burns, sunburn, trigeminal neuralgia, pain caused by colic, as well as spasms of the gastro-intestinal tract or uterus;
(b) visceral pain such as e.g. chronic pelvic pain, gynaecological pain, pain before and during menstruation, pain caused by pancreatitis, peptic ulcers, interstitial cystitis, renal colic, angina pectoris, pain caused by irritable bowel including Crohn's disease and ulcerative colitis, non-ulcerative dyspepsia and gastritis, prostatitis, non-cardiac thoracic pain and pain caused by myocardial ischaemia and cardiac infarct, pain caused by colic, nephritis and uveitis;
(c) neuropathic pain such as e.g. painful neuropathies, back pain, pain of diabetic neuropathy, pain after stroke, AIDS-associated neuropathic pain, Herpes zoster-induced pain, pain of lumbago, non-herpes-associated neuralgia, post-zoster neuralgia, nerve damage, cerebro-cranial trauma, pain of nerve damage caused by toxins or chemotherapy, in phantom pain, pain of multiple sclerosis, nerve root tears and painful traumatically-caused damage to individual nerves in carpal tunnel syndrome, in ulnar neuropathy, in radiculopathy, in hyperalgesia or allodynia associated pain;
(d) inflammatory/pain receptor-mediated pain in connection with diseases such as osteoarthritis, rheumatoid arthritis, rheumatic fever, tendo-synovitis, tendonitis, gout, vulvodynia, damage to and diseases of the muscles and fascia (muscle injury, fibromyalgia), atopic dermatitis, psoriasis, eczema, cerebral oedema, angiooedema, Crohn's disease, pelvitis, juvenile arthritis, spondylitis, gout-arthritis, psoriasis-arthritis, fibromyalgia, myositis, migraine, dental disease, influenza and other virus infections such as colds, bacterial infections, tensions, contusions, sprains, fractures or systemic lupus erythematodes,
(e) tumour pain associated with cancers such as lymphatic or myeloid leukaemia, Hodgkin's disease, non-Hodgkin's lymphomas, lymphogranulomatosis, lymphosarcomas, solid malignant tumours and extensive metastases;
(f) headache diseases such as e.g. headache of various origins, cluster headaches, migraine (with or without aura) and tension headaches.

The compounds are also suitable for treating

(g) inflammatory changes connected with diseases of the airways such as bronchial asthma, including allergic asthma (atopic and non-atopic) as well as bronchospasm on exertion, occupationally induced asthma, viral or bacterial exacerbation of an existing asthma and other non-allergically induced asthmatic diseases;
chronic obstructive pulmonary disease (COPD) including pulmonary emphysema, acute adult respiratory distress syndrome (ARDS), bronchitis, lung inflammation, cystic fibrosis, allergic rhinitis (seasonal and all year round) and sinusitis, non-allergic sinusitis, vasomotor rhinitis and diseases caused by dust in the lungs such as aluminosis, anthracosis, asbestosis, chalicosis, siderosis, silicosis, tabacosis and byssinosis;
(h) inflammatory phenomena caused by sunburn and burns, oedema after burns trauma, cerebral oedema and angiooedema, intestinal complaints including Crohn's diseases and ulcerative colitis, irritable bowel syndrome, pancreatitis, nephritis, cystitis (interstitial cystitis), uveitis; inflammatory skin diseases (such as e.g. angioderma, psoriasis and eczema), vascular diseases of the connective tissue, lupus, sprains and fractures;
(i) diabetes mellitus and its effects (such as e.g. diabetic vasculopathy, diabetic neuropathy, diabetic retinopathy) and diabetic symptoms in insulitis (e.g. hyperglycaemia, diuresis, proteinuria and increased renal excretion of nitrite and kallikrein);
(j) neurodegenerative diseases such as Parkinson's disease and Alzheimer's disease;
(k) sepsis and septic shock after bacterial infections or after trauma;
(l) syndromes that cause itching and allergic skin reactions;
(m) osteoporosis;
(n) epilepsy;
(o) damage to the central nervous system;
(p) wounds and tissue damage;
(q) inflammation of the gums;
(r) benign prostatic hyperplasia and hyperactive bladder;
(s) pruritus;
(t) vitiligo;
(u) disorders of the motility of respiratory, genito-urinary, gastro-intestinal or vascular regions,
(v) post-operative fever,
(w) obesity
(x) cardiovascular diseases such as atherosclerosis, cardiac insufficiency, heart valve diseases, for regulating blood pressure in hyper- or hypotension, or for the treatment of pulmonary arterial hypertension
(y) neuropsychatric diseases such as drug abuse, depression, schizophrenia or anxiety states and
(z) sleep disorders such as e.g. insomnia.

In addition to being suitable as human therapeutic agents, these substances are also useful in the veterinary treatment of domestic animals, exotic animals and farm animals.

### Combinations

For treating pain, it may be advantageous to combine the compounds according to the invention with stimulating substances such as caffeine or other pain-alleviating active compounds. If active compounds suitable for treating the cause of the pain are available, these can be combined with the compounds according to the invention. If, independently of the pain treatment, other medical treatments are also indicated, for example for high blood pressure or diabetes, the active compounds required can be combined with the compounds according to the invention.

The following compounds may be used for combination therapy, for example:

1. Non-steroidal antirheumatics (NSAR): including COX inhibitors such as propionic acid derivatives (alminoprofen, benoxaprofen, bucloxic acid, carprofen, fenhufen, fenoprofen, fiuprofen, fiulbiprofen, ibuprofen, indoprofen, ketoprofen, miroprofen, naproxen, oxaprozin, pirprofen, pranoprofen, suprofen, tiaprofenic acid, tioxaprofen), acetic acid derivatives (indomethacin, acemetacin, alclofenac, clidanac, diclofenac, fenflofenac, fentiazac, furofenac, ibufenac, isoxepac, oxpinax, sulindac, tiopinac, tolmetin, zidometacin, zomepirac), fenamic derivatives (meclofenamic acid, mefenamic acid, tolfenamic acid), biphenyl-carboxylic acid derivatives, oxicams (isoxicam, meloxicam, piroxicam, sudoxicam and tenoxicam), salicylic acid derivatives (acetylsalicylic acid, sulphasalazine, mesalazine, and olsalazine), pyrazolone (apazone, bezpiperylone, feprazone, mofebutazone, oxyphenbutazone, phenylbutazone, propyphenazone and metamizol), and coxibs (celecoxib, valdecoxib, rofecoxib, etoricoxib).
2. Opiate receptor agonists such as e.g. morphine, propoxyphen (Darvon), tramadol, buprenorphine.
3. Cannabinoid agonists such as e.g. GW-1000, KDS-2000, SAB-378, SP-104, NVP001-GW-843166, GW-842166X, PRS-211375.
4. Sodium channel blockers such as e.g. carbamazepine, mexiletin, lamotrigin, pregabalin, tectin, NW-1029, CGX-1002.
5. N-type calcium channel blockers such as e.g. ziconitide, NMED-160, SP1-860.
6. Serotonergic and noradrenergic modulators such as e.g. SR-57746, paroxetine, duloxetine, clonidine, amitriptyline, citalopram, flibanserin.
7. Corticosteroids such as e.g. betamethasone, budesonide, cortisone, dexamethasone, hydrocortisone, methylprednisolone, prednisolone, prednisone and triamcinolone.
8. Histamine H1-receptor antagonists such as e.g. bromopheniramine, chloropheniramine, dexchlorpheniramine, triprolidine, clemastine, diphenhydramine, diphenylpyraline, tripelennamine, hydroxyzine, methdilazine, promethazine, trimeprazine azatadine, cyproheptadine, antazoline, pheniramine, pyrilamine, astemizole, terfenadine, loratadine, cetirizine, desloratadine, fexofenadine, levocetirizine.
9. Histamine H2-receptor antagonists such as e.g. cimetidine, famotidine, and ranitidine.
10. Proton pump inhibitors such as e.g. omeprazole, pantoprazole, esomeprazole.
11. Leukotriene antagonists and 5-lipoxygenase inhibitors such as e.g. zafirlukast, montelukast, pranlukast and zileuton.
12. Local anaesthetics such as e.g. lidocaine, ambroxol.
13. VR1 agonists and antagonists such as e.g. NGX-4010, WL-1002, ALGRX-4975, WL-10001, AMG-517.
14. Nicotine receptor agonists such as e.g. ABT-202, A-366833, ABT-594, BTG-102, A-85380, CGX1204.
15. P2X3-receptor antagonists such as e.g. A-317491, ISIS-13920, AZD-9056.
16. NGF agonists and antagonists such as e.g. RI-724, RI-1024, AMG-819, AMG-403, PPH 207.
17. NK1 and NK2 antagonists such as e.g. DA-5018, R-116301, CP-728663, ZD-2249.
18. NMDA antagonists such as e.g. NER-MD-11, CNS-5161, EAA-090, AZ-756, CNP-3381.
19. Potassium channel modulators such as e.g. CL-888, ICA-69673, retigabin.
20. GABA modulators such as e.g. lacosamide.
21. Anti-migraine drugs such as e.g. sumatriptan, zolmitriptan, naratriptan, eletriptan, telcagepant.
22. iNOS inhibitors such as e.g. GSK 274150.
23. CCR2 antagonists such as e.g. PF-4136309, BMS-741672.
24. Anticonvulsants such as e.g. pregabalin or gabapentin.

The dosage necessary for obtaining a pain-alleviating effect is, in the case of intravenous administration, expediently from 0.01 to 3 mg/kg of body weight, preferably from 0.1 to 1 mg/kg, and, in the case of oral administration, from 0.1 to 8 mg/kg of body weight, preferably from 0.5 to 3 mg/kg, in each case 1 to 3 times per day. The compounds prepared according to the invention can be administered intravenously, subcutaneously, intramuscularly, intrarectally, intranasally, by inhalation, transdermally or orally, aerosol formulations being particularly suitable for inhalation. They can be incorporated into customary pharmaceutical preparations, such as tablets, coated tablets, capsules, powders, suspensions, solutions, metered-dose aerosols or suppositories, if appropriate together with one or more customary inert carriers and/or diluents, for example with maize starch, lactose, cane sugar, microcrystalline cellulose, magnesium stearate, polyvinylpyrrolidone, citric acid, tartaric acid, water, water/ethanol, water/glycerol, water/sorbitol, water/polyethylene glycol, propylene glycol, cetylstearyl alcohol, carboxymethylcellulose or fatty substances, such as hardened fat, or suitable mixtures thereof.

### Experimental Section

Generally, there are ¹H NMR and mass spectra for the compounds that were prepared. The ratios given for the eluants are in volume units of the solvents in question. For ammonia, the given volume units are based on a concentrated solution of ammonia in water.

Unless indicated otherwise, the acid, base and salt solutions used for working up the reaction solutions are aqueous systems having the stated concentrations. For chromatographic purification, silica gel from Millipore (MATREX™, 35 to 70 μm) or Alox (E. Merck, Darmstadt, Alumina 90 standardized, 63 to 200 μm, article No. 1.01097.9050) was used.

In the descriptions of the experiments, the following abbreviations are used:
CDI 1,1′-carbonyldiimidazole
TLC thin layer chromatography
DIPEA diisopropylethylamine
DMAP 4-dimethylaminopyridine
DMF dimethylformamide
DMSO dimethylsulphoxide
HATU O-(7-azabenzotriazol-1-yl)-N,N,N′,N′-tetramethyluronium hexafluorophosphate
tert tertiary
TBTU 2-(1H-benzotriazol-1-yl)-1.1.3.3-tetramethyluronium-tetrafluoroborate
THF tetrahydrofuran

The following analytical HPLC methods were used:

### Method 1: Column: Zorbax Stable Bond C18, 3.5 μM, 4.6×75 mm

Detection: 230-360 nm
Eluant A: water/0.1% formic acid
Eluant B: acetonitrile/0.1% formic acid
Gradient:

| time in min | % A | % B | flow rate in mL/min |
|---|---|---|---|
| 0.0 | 95.0 | 5.0 | 1.6 |
| 0.1 | 95.0 | 5.0 | 1.6 |
| 4.5 | 10.0 | 90.0 | 1.6 |
| 5.09 | 10.0 | 90.0 | 1.6 |
| 5.5 | 90.0 | 10.0 | 1.6 |

### Method 2: Column: Interchim Strategy C18, 5 μM, 4.6×50 mm

Detection: 220-320 nm
Eluant A: water/0.1% TFA
Eluant B: acetonitrile
Gradient:

| time in min | % A | % B | flow rate in mL/min |
|---|---|---|---|
| 0.0 | 95.0 | 5.0 | 3.0 |
| 0.3 | 95.0 | 5.0 | 3.0 |
| 2.0 | 2.0 | 98.0 | 3.0 |
| 2.4 | 2.0 | 98.0 | 3.0 |
| 2.45 | 95.0 | 5.0 | 3.0 |
| 2.8 | 95.0 | 5.0 | 3.0 |

### Method 3: Column: Merck Chromolith™ Flash RP18e, 4.6×25 mm

Detection: 210-400 nm
Eluant A: water/0.1% TFA
Eluant B: acetonitrile/0.1% TFA
Gradient:

| time in min | % A | % B | flow rate in mL/min |
|---|---|---|---|
| 0.0 | 95.0 | 5.0 | 2.5 |
| 0.2 | 95.0 | 5.0 | 2.5 |
| 1.5 | 2.0 | 98.0 | 2.5 |
| 1.7 | 2.0 | 98.0 | 2.5 |
| 1.9 | 95.0 | 5.0 | 2.5 |
| 2.2 | 95.0 | 5.0 | 2.5 |

### Method 4: Column: YMC-Pack ODS-AQ, 3.0 μM, 4.6×75 mm

Detection: 230-360 nm
Eluant A: water/0.1% formic acid
Eluant B: acetonitrile/0.1% formic acid
Gradient:

| time in min | % A | % B | flow rate in mL/min |
|---|---|---|---|
| 0.0 | 95.0 | 5.0 | 1.6 |
| 4.5 | 10.0 | 90.0 | 1.6 |
| 5.0 | 10.0 | 90.0 | 1.6 |
| 5.5 | 90.0 | 10.0 | 1.6 |

### Preparation of the End Compounds

### Example 1

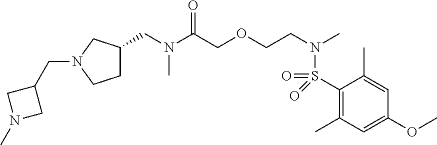

{2-[(4-methoxy-2,6-dimethyl-benzenesulphonyl)-methyl-amino]-ethoxy}-acetic acid (150 mg, 0.45 mmol), TBTU (151 mg, 0.45 mmol) and diisopropylethylamine (146 mg, 1.13 mmol) were dissolved in 3 ml DMF and stirred for 10 minutes at ambient temperature. Then [1-(1-methyl-azetidin-3-ylmethyl)-pyrrolidin-3-ylmethyl]-methylamine (89 mg, 0.45 mmol) was added and the mixture was stirred further overnight. The solvent was then evaporated off and the crude product thus obtained was purified by chromatography.

Yield: 22% of theory

C₂₅H₄₂N₄O₅S×CF₃COOH (624.7)

[M+H]+=511

HPLC (method 3): retention time=1.28 min

### Example 2

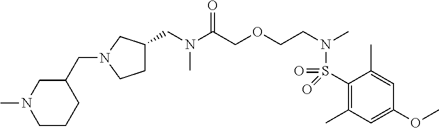

Prepared analogously to Example 1 from {2-[(4-methoxy-2,6-dimethyl-benzenesulphonyl)-methyl-amino]-ethoxy}-acetic acid and [1-(1-methyl-piperidin-3-ylmethyl)-pyrrolidin-3-ylmethyl]-methylamine.

Yield: 16% of theory

### C₂₇H₄₆N₄O₅S (538.7)

### [M+H]+=539

HPLC (method 3): retention time=1.30 min

### Example 3

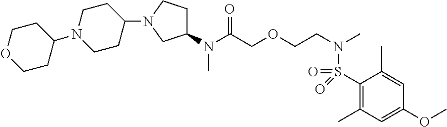

Prepared analogously to Example 1 from {2-[(4-methoxy-2,6-dimethyl-benzenesulphonyl)-methyl-amino]-ethoxy}-acetic acid and {1-[1-(tetrahydropyran-4-yl)-piperidin-4-yl]-pyrrolidin-3-yl}-methylamine.

Yield: 42% of theory

C₂₉H₄₈N₄O₆S×2HCl (653.7)

[M+H]+=581

HPLC (method 4): retention time=2.36 min

### Example 4

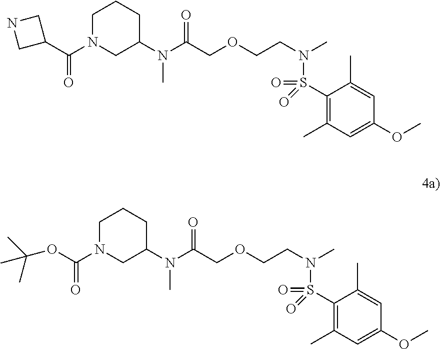

Prepared analogously to Example 1 from {2-[(4-methoxy-2,6-dimethyl-benzenesulphonyl)-methyl-amino]-ethoxy}-acetic acid and tert-butyl 3-methylamino-piperidine-1-carboxylate.

Yield: 98% of theory

C₂₅H₄₁N₃O₇S (527.7)

[M+H]+=528

HPLC (method 4): retention time=2.47 min

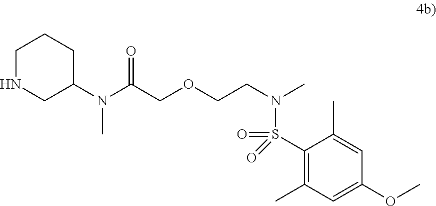

Tert-butyl 3-[(2-{2-[(4-methoxy-2,6-dimethyl-benzenesulphonyl)-methyl-amino]-ethoxy}-acetyl)-methyl-amino]-piperidine-1-carboxylate (product of Example 4a, 1.1 g, 1.97 mmol) was dissolved in 10 mL dichloromethane, combined with 6.5 mL trifluoroacetic acid and stirred for one hour at ambient temperature. Then the mixture was evaporated to dryness in vacuo, the residue was dissolved in 50 mL ethyl acetate, extracted three times with 25 mL 1M sodium hydroxide solution and once with 25 mL saturated sodium chloride solution. The combined aqueous extracts were extracted three times with 30 mL ethyl acetate and the combined organic extracts were then dried on magnesium sulphate and evaporated down.

Yield: 99% of theory

C₂₀F₃₃N₃O₅S (427.6)

[M+H]+=528

HPLC (method 3): retention time=1.49 min

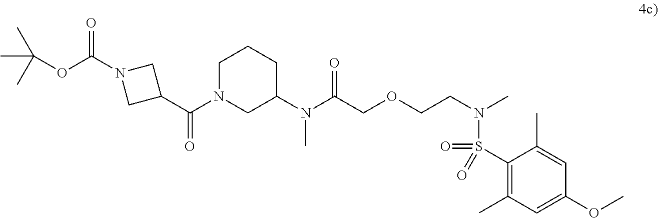

Prepared analogously to Example 1 from 2-{2-[(4-methoxy-2,6-dimethyl-benzenesulphonyl)-methyl-amino]-ethoxy}-N-methyl-N-piperidin-3-yl-acetamide (product of Example 4b) and 1-tert-butyloxycarbonyl-azetidine-3-carboxylic acid.

Yield: 96% of theory

C₂₉H₄₆N₄O₈S (610.8)

HPLC (method 3): retention time=2.28 min.

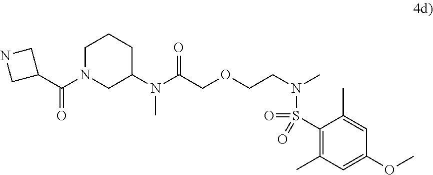

Tert-butyl 3-{3-[(2-{2-[(4-methoxy-2,6-dimethyl-benzenesulphonyl)-methyl-amino]-ethoxy}-acetyl)-methyl-amino]-piperidine-1-carbonyl}-azetidine-1-carboxylate (product of Example 4c, 217 mg, 0.36 mmol) was dissolved in 3 mL dichloromethane, combined with 2.5 mL trifluoroacetic acid and stirred for three hours at ambient temperature. Then the reaction mixture was evaporated to dryness and the crude product thus obtained was purified by chromatography.

Yield: 85% of theory

C₂₄H₃₈N₄O₆S×CF₃COOH (624.7)

[M+H]+=511

HPLC (method 3): retention time=1.55 min

### Example 5

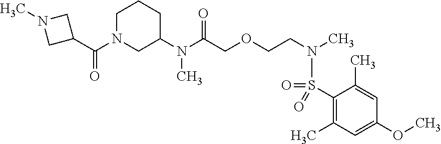

N-[1-(azetidine-3-carbonyl)-piperidin-3-yl]-2-{2-[(4-methoxy-2,6-dimethylbenzenesulphonyl)-methyl-amino]-ethoxy}-N-methyl-acetamide (product of Example 4, 293 mg, 0.57 mmol) was dissolved in 3 mL dioxane, then 0.13 mL of a 37% aqueous formaldehyde solution was added and the mixture was stirred for 30 minutes at ambient temperature. Then 304 mg (1.44 mmol) of sodium triacetoxyborohydride were added and the mixture was stirred for a further 5 hours at ambient temperature. Then the reaction mixture was evaporated to dryness and the crude product thus obtained was purified by chromatography.

Yield: 6.3% of theory

C₂₅H₄₀N₄O₆S (524.7)

[M+H]+=525

HPLC (method 3): retention time=1.56 min

### Example 6

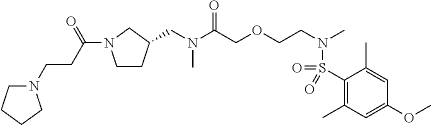

Prepared analogously to Example 1 from {2-[(4-methoxy-2,6-dimethyl-benzenesulphonyl)-methyl-amino]-ethoxy}-acetic acid and 1-(3-methylaminomethyl-pyrrolidin-1-yl)-3-pyrrolidin-1-yl-propan-1-one.

Yield: 21% of theory

C₂₇H₄₄N₄O₆S (552.7)

[M+H]+=553

HPLC (method 3): retention time=1.62 min

### Example 7

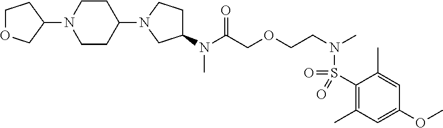

Prepared analogously to Example 1 from {2-[(4-methoxy-2,6-dimethyl-benzenesulphonyl)-methyl-amino]-ethoxy}-acetic acid and methyl-{1-[1-(tetrahydrofuran-3-yl)-piperidin-4-yl]-pyrrolidin-3-yl}-amine.

Yield: 42% of theory

C₂₈H₄₆N₄O₆S×2HCl (639.7)

[M+H]+=567

HPLC (method 4): retention time=2.99 min

### Example 8

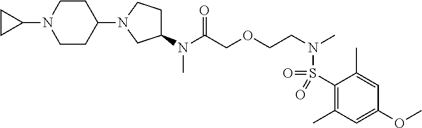

Prepared analogously to Example 1 from {2-[(4-methoxy-2,6-dimethyl-benzenesulphonyl)-methyl-amino]-ethoxy}-acetic acid and [1-(1-cyclopropyl-piperidin-4-yl)-pyrrolidin-3-yl]-methyl-amine.

Yield: 46% of theory

C₂₇H₄₄N₄O₅S×2HCl (609.7)

[M+H]+=537

HPLC (method 1): retention time=3.10 min

### Example 9

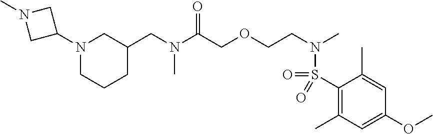

Prepared analogously to Example 1 from {2-[(4-methoxy-2,6-dimethyl-benzenesulphonyl)-methyl-amino]-ethoxy}-acetic acid and methyl-[1-(1-methyl-azetidin-3-yl)-piperidin-3-yl-methyl]-amine.

Yield: 76% of theory

C₂₅H₄₂N₄O₅S×CF₃COOH (624.7)

[M+H]+=511

HPLC (method 3): retention time=1.34 min

### Example 10

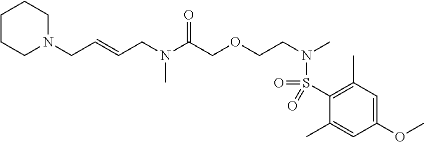

Prepared analogously to Example 1 from {(2-[(4-methoxy-2,6-dimethyl-benzenesulphonyl)-methyl-amino]-ethoxy}-acetic acid and methyl-(4-piperidin-1-yl)-but-2-enyl)-amine.

Yield: 92% of theory

C₂₄H₃₉N₃O₅S×CF₃COOH (595.7)

[M+H]+=482

HPLC (method 2): retention time=1.37 min

### Example 11

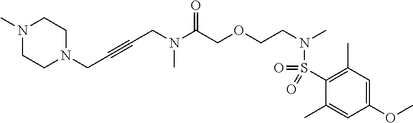

Prepared analogously to Example 1 from {2-[(4-methoxy-2,6-dimethyl-benzenesulphonyl)-methyl-amino]-ethoxy}-acetic acid and methyl-4-[(4-methyl-piperazin-1-yl)-but-2-ynyl]-amine.

Yield: 43% of theory

C₂₄H₃₈N₄O₅S×CF₃COOH (608.7)

[M+H]+=495

HPLC (method 2): retention time=1.27 min

### Example 12

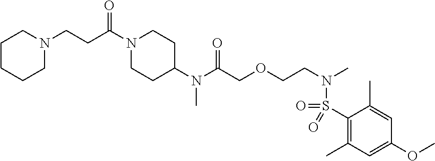

Prepared analogously to Example 1 from {2-[(4-methoxy-2,6-dimethyl-benzenesulphonyl)-methyl-amino]-ethoxy}-acetic acid and 1-(4-methylamino-piperidin-1-yl)-3-piperidin-1-yl-propan-1-one.

Yield: 77% of theory

C₂₈H₄₆N₄O₆S×CF₃COOH (680.8)

[M+H]+=567

HPLC (method 3): retention time=1.61 min

### Example 13

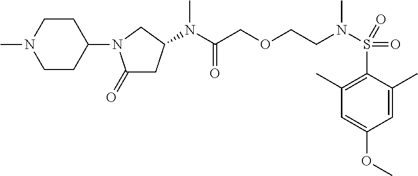

Prepared analogously to Example 1 from {2-[(4-methoxy-2,6-dimethyl-benzenesulphonyl)-methyl-amino]-ethoxy}-acetic acid and 4-methylamino-1-(1-methyl-piperidin-4-yl)-pyrrolidin-2-one.

Yield: 19% of theory

C₂₅H₄₀N₄O₆S×CF₃COOH (638.7)

[M+H]+=525

HPLC (method 3): retention time=1.66 min

The following Examples describe pharmaceutical formulations which contain as active substance any desired compound of general formula I:

### Example I

### Dry Ampoule with 75 mg of Active Compound Per 10 ml

### Composition:

| | | |
|---|---|---|
| Active compound | 75.0 | mg |
| Mannitol | 500 | mg |
| Water for injection | 10.0 | ml |

### Production:

Active compound and mannitol are dissolved in water. The charged ampoules are freeze dried. Water for injection is used to dissolve to give the solution ready for use.

### Example II

### Tablet with 50 mg of Active Compound

### Composition:

| | |
|---|---|
| (1) Active compound | 50.0 mg |
| (2) Lactose | 98.0 mg |
| (3) Maize starch | 50.0 mg |
| (4) Polyvinylpyrrolidone | 15.0 mg |
| (5) Magnesium stearate | 2.0 mg |
| | 215.0 mg |

### Production:

(1), (2) and (3) are mixed and granulated with an aqueous solution of (4). (5) is admixed to the dry granules. Tablets are compressed from this mixture, biplanar with a bevel on both sides and dividing groove on one side.

Diameter of the tablets: 9 mm.

### Example III

### Tablet with 350 mg of Active Compound

### Composition:

| | | |
|---|---|---|
| (1) Active compound | 350.0 | mg |
| (2) Lactose | 136.0 | mg |
| (3) Maize starch | 80.0 | mg |
| (4) Polyvinylpyrrolidone | 30.0 | mg |
| (5) Magnesium stearate | 4.0 | mg |
| | 600.0 | mg |

### Production:

(1), (2) and (3) are mixed and granulated with an aqueous solution of (4). (5) is admixed to the dry granules. Tablets are compressed from this mixture, biplanar with a bevel on both sides and dividing groove on one side.

Diameter of the tablets: 12 mm.

### Example IV

### Capsule with 50 mg of Active Compound

### Composition:

| | |
|---|---|
| (1) Active compound | 50.0 mg |
| (2) Maize starch dried | 58.0 mg |
| (3) Lactose powdered | 50.0 mg |
| (4) Magnesium stearate | 2.0 mg |
| | 160.0 mg |

### Production:

(1) is triturated with (3). This trituration is added to the mixture of (2) and (4) with vigorous mixing.

This powder mixture is packed into hard gelatine two-piece capsules of size 3 in a capsule-filling machine.

### Example V

### Capsules with 350 mg of Active Compound

### Composition:

| | | |
|---|---|---|
| (1) Active compound | 350.0 | mg |
| (2) Maize starch dried | 46.0 | mg |
| (3) Lactose powdered | 30.0 | mg |
| (4) Magnesium stearate | 4.0 | mg |
| | 430.0 | mg |

### Production:

(1) is triturated with (3). This trituration is added to the mixture of (2) and (4) with vigorous stirring.

This powder mixture is packed into hard gelatine two-piece capsules of size 0 in a capsule-filling machine.

### Example VI

### Suppositories with 100 mg of Active Compound

1 suppository comprises:

| | |
|---|---|
| Active compound | 100.0 mg |
| Polyethylene glycol (M.W. 1500) | 600.0 mg |
| Polyethylene glycol (M.W. 6000) | 460.0 mg |
| Polyethylene sorbitan monostearate | 840.0 mg |
| | 2000.0 mg |

1. A compound of the formula I 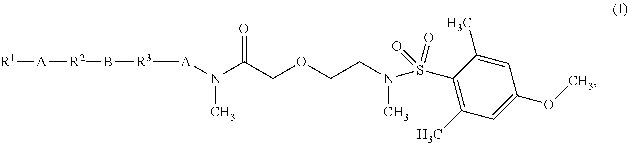
wherein
A denotes
(a) a bond or
(b) a C₁₋₃-alkylene group,
B denotes
(a) a bond,
(b) a C₁₋₃-alkylene or
(c) a —C(O)— group,
R¹ denotes
(a) H,
(b) C₁₋₃-alkyl,
(c) C₃₋₆-cycloalkyl,
(d) a 5- or 6-membered, saturated aza-heterocycle or
(e) a 5- or 6-membered, saturated oxa-heterocycle,
R² denotes
(a) C₁₋₃-alkylene,
(b) a 4- to 6-membered, saturated aza-heterocycle, or
(c) a 6- to 7-membered, saturated diaza-heterocycle,
R³ denotes
(a) C₂₋₄-alkenylene,
(b) C₂₋₄-alkynylene or
(c) a 5- to 6-membered, saturated aza-heterocycle, wherein additionally a methylene group may be substituted by a carbonyl group,
or a salt thereof.

2. A compound of the formula I according to claim 1, wherein
A denotes a bond or a —CH₂— group,
B denotes a bond, a —CH₂— or —C(O)— group,
R¹ denotes H, H₃C or a group selected from 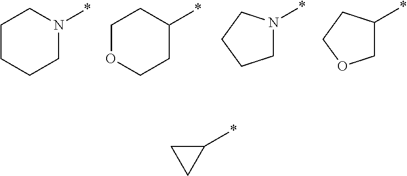
R² denotes a group selected from 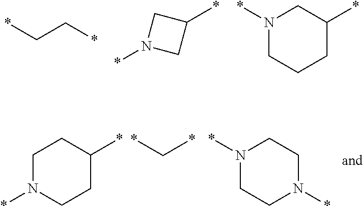
R³ denotes a group selected from 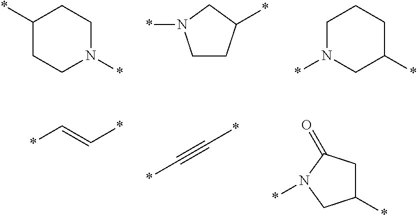
or a salt thereof.

3. A compound according to claim 1 selected from the group consisting of:

| Example | Structure |
|---|---|
| (1) | 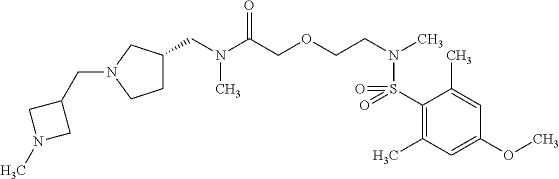 |
| (2) | 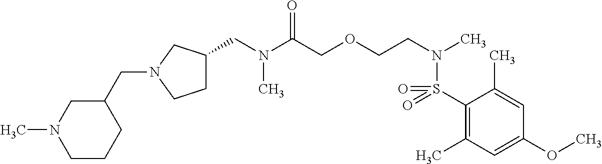 |
| (3) | 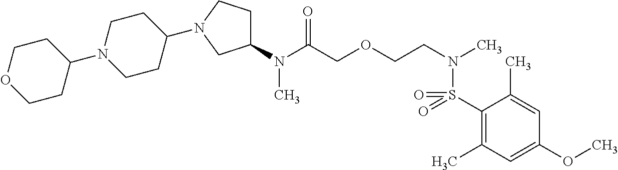 |
| (4) | 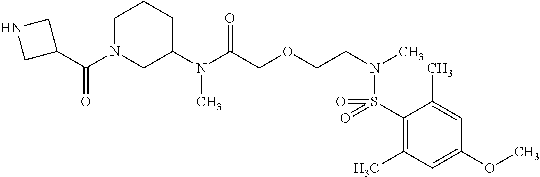 |
| (5) | 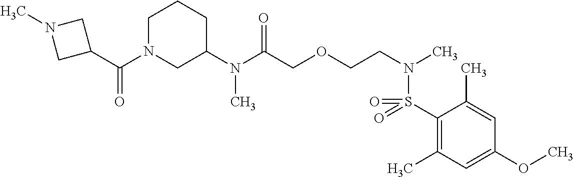 |
| (6) | 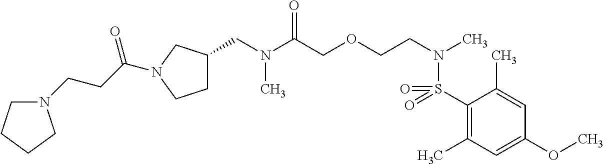 |
| (7) | 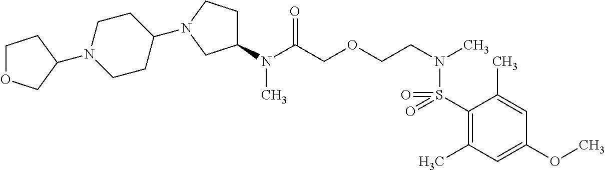 |
| (8) | 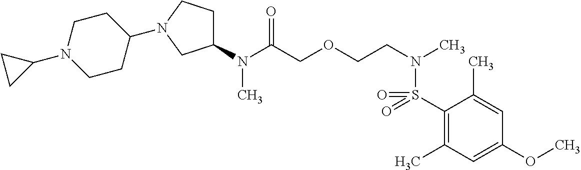 |
| (9) | 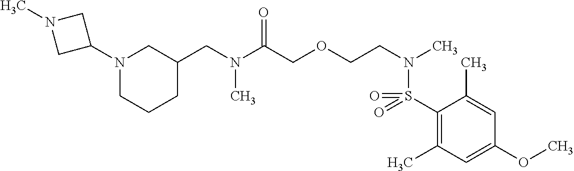 |
| (10) | 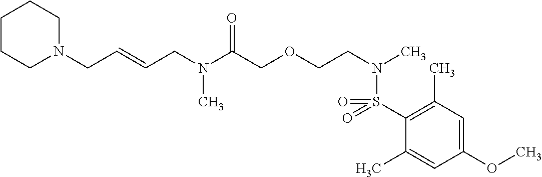 |
| (11) | 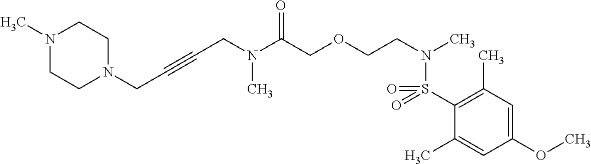 |
| (12) | 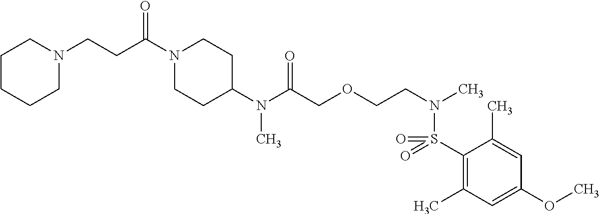 |

and

| | |
|---|---|
| (13) | 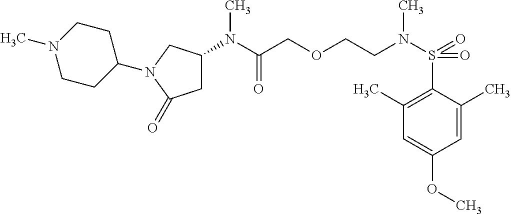 |

or a salt thereof.

4. A physiologically acceptable salt of a compound according to claim 1, 2 or 3.

5. A pharmaceutical composition comprising a compound according to claim 1, 2, or 3 or a physiologically acceptable salt thereof together with one or more inert carriers and/or diluents.

6. A method for treating acute pain, visceral pain, neuropathic pain, inflammatory/pain receptor mediated pain, tumor pain and headache diseases which method comprising administering to a host suffering from the same a therapeutically effective amount of a compound according to claim 1, 2 or 3 or a physiologically acceptable salt thereof.

7. (canceled)
